(19) United States
(12) Patent Application Publication
(10) Pub. No.: US 2023/0302441 A1
(43) Pub. Date: Sep. 28, 2023

(54) WELL DEFINED QUATERNARY AMMONIUM FUNCTIONALIZED QUATERPHENYLENE POLYMER DERIVATIVES FOR LOW AND HIGH TEMPERATURE POLYMER ELECTROLYTE MEMBRANE FUEL CELLS

(71) Applicant: Advent Technologies Holdings, Inc., Boston, MA (US)

(72) Inventors: Christos L. Chochos, Ilion (GR); Panagiotis Bexis, Patras (GR); Konstantinos Gournas, Patras (GR); George Paloumbis, Patra (GR); Vasilis Gregoriou, Drosia Attica (GR)

(21) Appl. No.: 17/704,711

(22) Filed: Mar. 25, 2022

(51) Int. Cl. B01J 41/14; C08F 238/00; C07C 49/697

(52) U.S. Cl. CPC B01J 41/14; C08F 238/00; C07C 49/697; C07C 2601/10

(57) ABSTRACT

The invention relates to the development of quaterphenylene polymer derivatives with synthetic methodologies towards controllable and integer degree of functionalization on the quaterphenylene polymer backbone with specific number of side phenyl groups and monovalent hydrocarbon groups terminated by a functional group consisting of quaternary ammonium groups resulting to precise IEC, water uptake and anionic conductivity without batch-to-batch variations and compatible with industrial manufacturing processes for use as membranes or ionomers in low temperature and high temperature polymer electrolyte membrane fuel cells.

### FIELD OF INVENTION

The invention relates to the fabrication of quaterphenylene polymer derivatives and membranes thereof with specific and controllable molecular characteristics, such as the side phenyl groups, and monovalent hydrocarbon groups terminated by a functional group consisting of quaternary ammonium groups for use as membranes or ionomers in low temperature and high temperature polymer electrolyte membrane fuel cells.

### DESCRIPTION OF THE RELATED ART

Fuel cells are promising devices for clean power generation in a variety of economically and environmentally significant applications. By using hydrogen produced from renewable energy sources, such as solar and wind, fuel cells can provide carbon-neutral power without any pollutants, such as SOX and NOR. Initial commercialization of clean, high-efficiency fuel cell electric vehicles is already underway, but further technological innovation is needed to improve cost-competitiveness of fuel cells in the market place.

Currently, there are two general types of fuel cells: low temperature fuel cells and high temperature fuel cells. Low-temperature proton exchange membrane (PEM) fuel cells utilizing Nafion® polymeric materials for membranes require a high level of hydration, which limits the operating temperature to less than 100° C. to preclude excessive water evaporation. The structure for Nafion® is provided below.

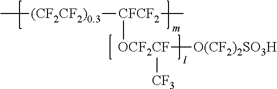

Low-temperature PEM fuel cells that use Nafion® are currently being commercialized in fuel cell vehicles, but these cells can operate only at relatively low temperatures and high hydration levels; therefore, they require humidified inlet streams and large radiators to dissipate waste heat.

In contrast, high-temperature PEM fuel cells typically utilize membranes comprising phosphoric acid-doped polybenzimidazole, shown below.

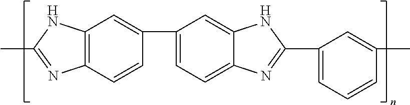

High temperature fuel cells can operate effectively up to 180° C.; however, these devices degrade when exposed to water below 140° C. High-temperature PEM fuel cells that use phosphoric acid (PA)-doped polybenzimidazole (PBI) could address these issues, but these PBI-based cells are difficult to operate below 140° C. without excessive loss of PA. The limited operating temperature range makes them unsuitable for automotive applications, where water condensation from frequent cold start-ups and oxygen reduction reaction at the fuel cell cathode occur during normal vehicle drive cycles.

Quaternary ammonium (QA) functionalized polymers are known, and some have been developed for alkaline electrochemical devices. As currently understood, phosphoric acid-doped QA functionalized polymers have been reported only once, by the Wegner research group at Max Planck Institute in 1999, (A. Bozkurt et al., Proton-conducting Polymer Electrolytes based on Phosphoric Acid, Solid State Ionics, 125, 225 (1999)). Bozkurt et al. used poly(diallyldimethylammonium) as the polymeric material used to produce the fuel cell membrane, and their approach was substantially the same as that of the PA-doped PBI in three respects: 1) the quaternary ammonium moiety of the synthesized polymer was located within the polymer backbone; 2) the quaternary ammonium moiety concentration was high (about 7.2 mmol/gram, which is comparable to that of PBI, about 6.5 mmol/gram); and 3) the researchers were primarily interested in anhydrous proton conductivity.

Currently, QA functionalized polyphenylenes are considered the state-of-the-art polymeric materials for low temperature and high temperature fuel cells, shown below.

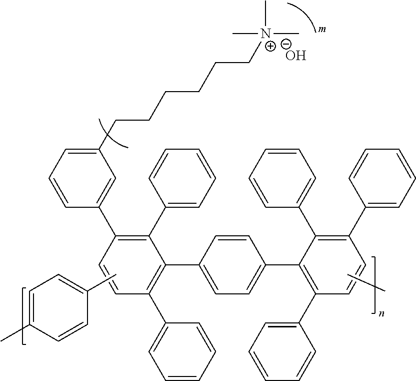

Due to the possibility of forming entirely aromatic backbone polymers (J. Stille, et al., Diels-Alder polymerizations: Polymers containing controlled aromatic segments, Journal of Polymer Science Part B:Polymer Letters, 4, 791 (1966)), poly(phenylene)s made by Diels-Alder polymerization (DAPP) have been demonstrated to be suitable for polymer electrolyte membranes. With the further QA functionalization DAPP-based ion exchange membrane was first utilized as anion exchange membranes before being utilized in high temperature polymer electrolyte membrane fuel cell (C. H. Fujimoto et al., Ionomeric Poly(phenylene) Prepared by Diels-Alder Polymerization: Synthesis and Physical Properties of a Novel Polyelectrolyte Macromolecules, 38, 5010 (2005)).

However, the QA functionalized polyphenylene shown in the above along with its precursor polymer derivatives are synthesized through a post functionalization process that is not controlled and can cause significant variations on the ion exchange capacity (IEC), water uptake, anionic conductivity and swelling properties from batch to batch. From industrial manufacturing point of view this can cause significant alternations to the performance of the final systems.

### SUMMARY OF THE INVENTION

The subject invention relates to the development of QA functionalized quaterphenylene polymer derivatives with synthetic methodologies towards controllable and exact degree of functionalization on the quaterphenylene polymer backbone resulting to specific IEC, water uptake and anionic conductivity without batch-to-batch variations and compatible with industrial manufacturing processes for use as membranes or ionomers in low temperature and high temperature polymer electrolyte membrane fuel cells.

Additional features and benefits of the present invention will become apparent from the detailed description, figures, and claims set forth below.

### BRIEF DESCRIPTION OF THE DRAWINGS

A more complete understanding of the invention may be attained by reference to the drawings, in which:

FIG. 1 is a schematic illustration of the synthetic procedure towards 2P2NQP according to the invention.

FIG. 2 is a schematic illustration of the synthetic procedure towards 4P2NQP, 4P3NQP and 4P4NQP according to the invention.

FIG. 3 is a schematic illustration of the synthetic procedure towards 6P2NQP, 6P3NQP and 6P4NQP according to the invention.

FIG. 4 is a schematic illustration of a fuel cell of the type in which the invention is practiced.

### DETAILED DESCRIPTION OF THE INVENTION

The present invention relates to QA functionalized quaterphenylene polymer derivatives with integer numbers of substituents on the quaterphenylene backbone resulting in specific IEC, water uptake and anionic conductivity without batch-to-batch variations and compatible with industrial manufacturing processes. The structures of the materials are given below.

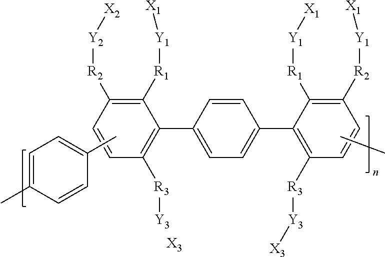

In the embodiment illustrated immediately above, the polymers are consisting of a quaterphenylene backbone with 150 to 300 repeat units.

In the embodiment, the polymers include either two (R₁=phenyl; R₂=R₃=hydrogen) or four (R₁=R₂=phenyl; R₃=hydrogen) or six (R₁=R₂=R₃=phenyl) side phenyl rings.

In the embodiment, when the polymer consists of two side phenyl rings (R₁=phenyl; R₂=R₃=hydrogen) it can include two side monovalent hydrocarbon groups including four to 21 carbon atoms that may be the same or different. The monovalent hydrocarbon groups may have a straight chain or a branched chain structure. A straight or branched chain of a monovalent hydrocarbon group may also be interrupted by O, N, or S atoms. The monovalent hydrocarbon groups can be terminated by a functional group consisting of chlorine, bromine, iodine or QA groups.

In the embodiment, when the polymer consists of four side phenyl rings (R₁=R₂=phenyl; R₃=hydrogen) it can include exact two, three or four side monovalent hydrocarbon groups including four to 21 carbon atoms that may be the same or different. The monovalent hydrocarbon groups may have a straight chain or a branched chain structure. A straight or branched chain of a monovalent hydrocarbon group may also be interrupted by O, N, or S atoms. The monovalent hydrocarbon groups can be terminated by a functional group consisting of chlorine, bromine, iodine or QA groups.

In the embodiment, when the polymer consists of six side phenyl rings (R₁=R₂=R₃=phenyl) it can include exact two, three or four side monovalent hydrocarbon groups including four to 21 carbon atoms that may be the same or different. The monovalent hydrocarbon groups may have a straight chain or a branched chain structure. A straight or branched chain of a monovalent hydrocarbon group may also be interrupted by O, N, or S atoms. The monovalent hydrocarbon groups can be terminated by a functional group consisting of chlorine, bromine, iodine or QA groups.

A functional group is selected to impart a desired property to a polyphenylene derivative, including rendering a functional group susceptible to substitution with another functional group. One example of a functional group is a functional group that is a cationic group. As noted above, polymers including pendant cationic groups have found use in anion exchange membranes and fuel cells. An example of a cationic group is an ammonium.

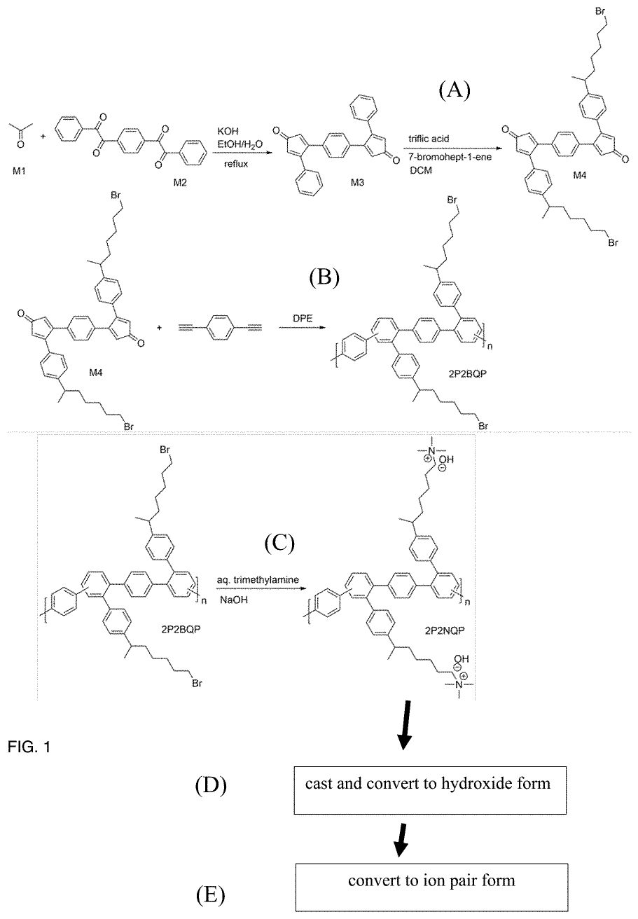

FIG. 1 shows an embodiment of the invention providing a method for the synthesis of such a polymer that consists of two side phenyl rings with exact two 7-bromoheptan-2-yl side chains. In this embodiment, in step (A), functional groups, each comprising one or more monovalent hydrocarbon groups terminated by a halogen, e.g., chlorine, bromine, or iodine, are first introduced to monomer precursors — and, more particularly, here, the exact two 7-bromoheptan-2-yl side chains are attached to 4,4′-(1,4-phenylene)bis(3-phenylcyclopenta-2,4-dien-1-one) (M3) using a Friedel-Crafts acylation reaction with catalytic amount of triflic acid and 2 equivalent 7-bromohept-1-ene resulting the 4,4′-(1,4-phenylene)bis(3-(4-(7-bromoheptan-2-yl)phenyl)cyclopenta-2,4-dien-1-one) (M4). The 4,4′-(1,4-phenyl ene)bis(3-phenylcyclopenta-2,4-dien-1-one) is prepared through a condensation reaction between propan-2-one (MI) and 2,2′-(1,4-phenylene)bis(1-phenylethane-1,2-dione) (M2).

Then, in step (B), the resulting monomer groups are polymerized—and, more particularly, here, the quaterphenylene polymer derivative 2P2BQP is synthesized by Diels Alder polymerization reaction where 4,4′-(1,4-phenylene)bis(3-(4-(7-bromoheptan- 2-yl)phenyl)cyclopenta-2,4-dien-1-one) (M4) reacts with p-bis(ethynyl)benzene to yield carbon monoxide and a polymer with a quaterphenylene backbone with a mix of meta and para configurations imparted by the selectivity of a Diels-Alder polymerization, two phenyl side rings and exactly two 7-bromoheptan-2-yl side groups per repeat unit.

Following the formation of 2P2BQP, in step (C), the halogen functional group is substituted with a nitrogen-containing base. FIG. 1 shows a quaterphenylene polymer derivative according to the invention including exactly two functional groups that is an ammonium group per repeat unit identified as 2P2NQP.

In some embodiments, in step (D), that quaterphenylene polymer derivative is cast in solvent to form a thin film, which is converted to the hydroxide form, all as is within the ken of those skilled in the art in view of the teachings hereof. The hydroxide form can then, in step (E), be converted to the ion pair form with phosphoric or phosphinic acids, as is also within the ken of those skilled in the art in view of the teachings hereof.

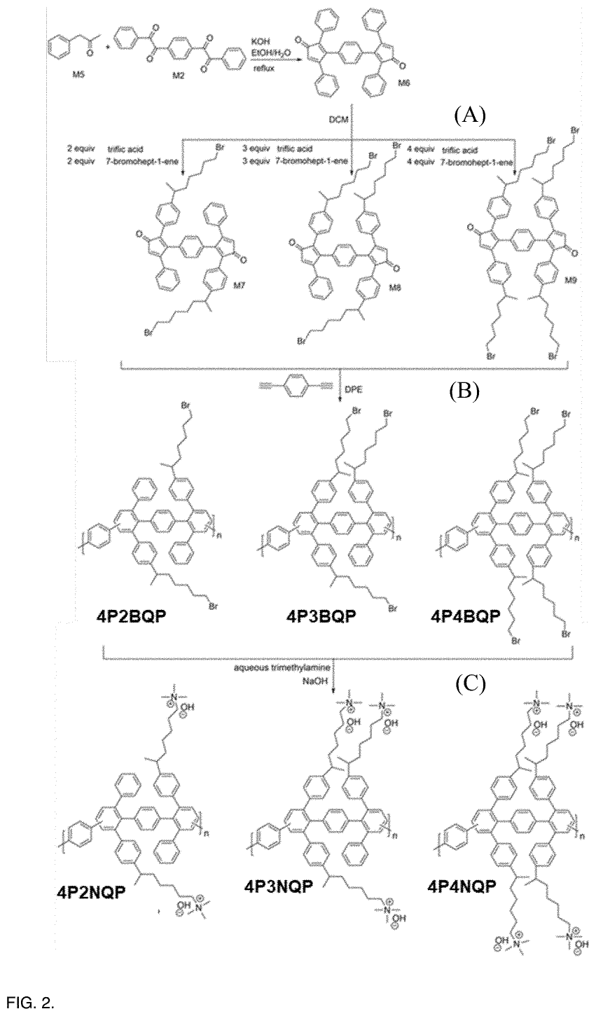

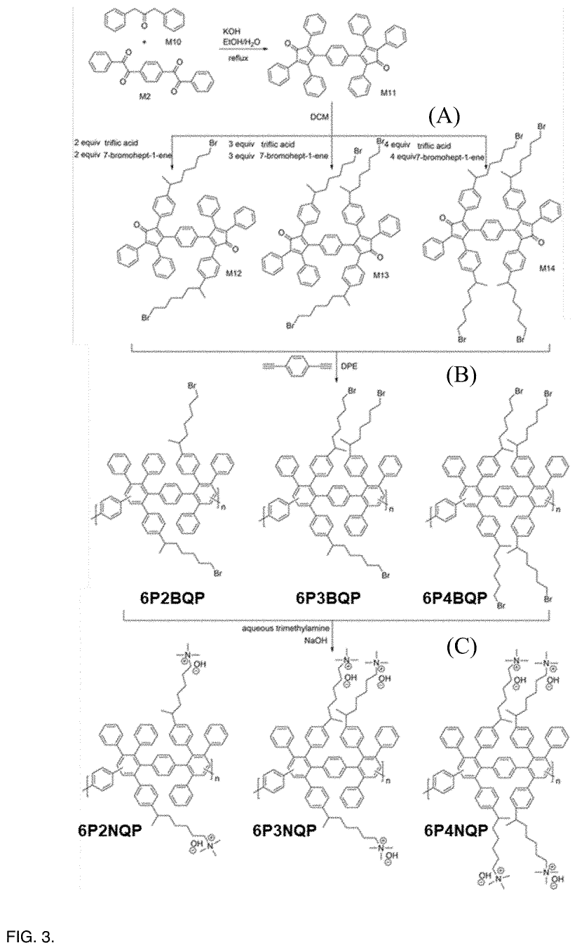

Like methods of synthesis, i.e., those involving steps (A)-(C) and, optionally, steps (D)-(E), can be practiced for the synthesis of quaterphenyl based polymers that consist of four side phenyl rings and for the synthesis of quaterphenyl based polymers consist of six side phenyl rings, as shown in FIGS. 2 and 3, respectively, and described in the accompanying text, below. For simplicity, only steps (A)-(C) are annotated in each of those latter drawings, though, it will be appreciated that steps (D)-(E) can be practiced with their respective embodiments as well.

FIG. 2 shows an embodiment of a method for the synthesis of quaterphenyl based polymers that consist of four side phenyl rings with exact two, three and four 7-bromoheptan-2-yl side chains. In the embodiment of that figure, the exact two 7-bromoheptan-2-yl side chains are attached to 3,3′-(1,4-phenylene)bis(2,4-diphenylcyclopenta-2,4-dien-1-one) (M6) using a Friedel-Crafts acylation reaction with catalytic amount of triflic acid and 2 equivalents of 7-bromohept-1-ene resulting the 3,3′-(1,4-phenyl ene)bis(2-(4-(7-bromoheptan-2-yl)phenyl)-4-phenylcyclopenta-2,4-dien-1-one) (M7). The 3,3′-(1,4-phenylene)bis(2,4-diphenylcyclopenta-2,4-dien-1-one) is prepared through a condensation reaction between 1-phenylpropan-2-one (M5) and 2,2′-(1,4-phenylene)bis(1-phenylethane-1,2-dione) (M2).

In the embodiment, the exact three 7-bromoheptan-2-yl side chains are attached to 3,3′-(1,4-phenylene)bis(2,4-diphenylcyclopenta-2,4-dien-1-one) (M6) using a Friedel-Crafts acylation reaction with catalytic amount of triflic acid and 3 equivalents of 7-bromohept-1-ene resulting the 3-(4-(2, 5-bis(4-(7-bromoheptan-2-yl)phenyl)-3-oxocyclopenta-1,4-dien-1-yl)phenyl)-2-(4-(7-bromoheptan-2-yl)phenyl)-4-phenylcyclopenta-2,4-dien-1-one (M8).

In the embodiment, the exact four 7-bromoheptan-2-yl side chains are attached to 3,3′-(1,4-phenylene)bis(2,4-diphenylcyclopenta-2,4-dien-1-one) (M6) using a Friedel-Crafts acylation reaction with catalytic amount of triflic acid and 4 equivalents of 7-bromohept-1-ene resulting the 3,3′-(1,4-phenylene)bis(2,4-bis(4-(7-bromoheptan-2-yl)phenyl)cyclopenta-2,4-dien-1-one) (M9).

In the embodiment, the quaterphenylene polymer derivative 4P2BQP is synthesized by Diets Alder polymerization reaction where 3,3′-(1,4-phenylene)bis(2-(4-(7-bromoheptan-2-yl)phenyl)-4-phenylcyclopenta-2,4-dien-1-one) (M7) reacts with p-bis(ethynyl)benzene to yield carbon monoxide and a polymer with a quaterphenylene backbone with a mix of meta and para configurations imparted by the selectivity of a Diels-Alder polymerization, four phenyl side rings and exactly two 7-bromoheptan-2-yl side groups per repeat unit.

In the embodiment, the quaterphenylene polymer derivative 4P3BQP is synthesized by Diets Alder polymerization reaction where 3-(4-(2,5-bis(4-(7-bromoheptan-2-yl)phenyl)-3-oxocyclopenta-1,4-dien-1-yl)phenyl)-2-(4-(7-bromoheptan-2-yl)phenyl)-4-phenylcyclopenta-2,4-dien-1-one (M8) reacts with p-bis(ethynyl)benzene to yield carbon monoxide and a polymer with a quaterphenylene backbone with a mix of meta and para configurations imparted by the selectivity of a Diels-Alder polymerization, four phenyl side rings and exactly three 7-bromoheptan-2-yl side groups per repeat unit.

In the embodiment, the quaterphenylene polymer derivative 4P4BQP is synthesized by Diets Alder polymerization reaction where 3,3′-(1,4-phenylene)bis(2,4-bis(4-(7-bromoheptan-2-yl)phenyl)cyclopenta-2,4-dien-1-one) (M9) reacts with p-bis(ethynyl)benzene to yield carbon monoxide and a polymer with a quaterphenylene backbone with a mix of meta and para configurations imparted by the selectivity of a Diels-Alder polymerization, four phenyl side rings and exactly four 7-bromoheptan-2-yl side groups per repeat unit.

In the embodiment, following the formation of 4P2BQP the bromine functional groups are substituted with a nitrogen-containing base. FIG. 2 shows a quaterphenylene polymer derivative including exactly two functional groups that is an ammonium group per repeat unit identified as 4P2NQP.

In the embodiment, following the formation of 4P3BQP the bromine functional groups are substituted with a nitrogen-containing base. FIG. 2 shows a quaterphenylene polymer derivative including exactly three functional groups that is an ammonium group per repeat unit identified as 4P3NQP.

In the embodiment, following the formation of 4P4BQP the bromine functional groups are substituted with a nitrogen-containing base. FIG. 2 shows a quaterphenylene polymer derivative including exactly four functional groups that is an ammonium group per repeat unit identified as 4P4NQP.

FIG. 3 shows an embodiment of a method for the synthesis of quaterphenyl based polymers consist of six side phenyl rings with exact two, three and four 7-bromoheptan-2-yl side chains. In the embodiment, the exact two 7-bromoheptan-2-yl side chains are attached to 4,4′-(1,4-phenylene)bis(2,3,5-triphenylcyclopenta-2,4-dien-1-one) (M11) using a Friedel-Crafts acylation reaction with catalytic amount of triflic acid and 2 equivalents of 7-bromohept-1-ene resulting the 4,4′-(1,4-phenylene)bis(5-(4-(7-bromoheptan-2-yl)phenyl)-2,3-diphenylcyclopenta-2,4-dien-1-one) (M12). The 4,4′-(1,4-phenylene)bis(2,3,5-triphenylcyclopenta-2,4-dien-1-one) is prepared through a condensation reaction between 1,3-diphenylpropan-2-one (M10) and 2,2′-(1,4-phenylene)bis(1-phenylethane-1,2-dione) (M2).

In the embodiment of FIG. 3, the exact three 7-bromoheptan-2-yl side chains are attached to 4,4′-(1,4-phenylene)bis(2,3,5-triphenylcyclopenta-2,4-dien-1-one) (M11) using a Friedel-Crafts acylation reaction with catalytic amount of triflic acid and 3 equivalents of 7-brom ohept-1-ene resulting the 3-(4-(2,5-bis(4-(7-brom oheptan- 2-yl)phenyl)-3-oxo-4-phenylcyclopenta-1,4-dien-1-yl)phenyl)-2-(4-(7-bromoheptan-2-yl)phenyl)-4,5-diphenylcyclopenta-2,4-dien-1-one (M13).

In the embodiment, the exact four 7-bromoheptan-2-yl side chains are attached to 4,4′-(1,4-phenylene)bis(2,3,5-triphenylcyclopenta-2,4-dien-1-one) (M11) using a Friedel-Crafts acylation reaction with catalytic amount of triflic acid and 4 equivalents of 7-bromohept-1-ene resulting the 4,4′-(1,4-phenylene)bis(3,5-bis(4-(7-bromoheptan-2-yl)phenyl)-2-phenylcyclopenta-2,4-dien-1-one) (M14).

In the embodiment, the quaterphenylene polymer derivative 6P2BQP is synthesized by Diets Alder polymerization reaction where 4,4′-(1,4-phenylene)bis(5-(4-(7-bromoheptan-2-yl)phenyl)-2,3-diphenylcyclopenta-2,4-dien-1-one) (M12) reacts with p-bis(ethynyl)benzene to yield carbon monoxide and a polymer with a quaterphenylene backbone with a mix of meta and para configurations imparted by the selectivity of a Diels-Alder polymerization, six phenyl side rings and exactly two 7-bromoheptan-2-yl side groups per repeat unit.

In the embodiment, the quaterphenylene polymer derivative 6P3BQP is synthesized by Diets Alder polymerization reaction where 3-(4-(2,5-bis(4-(7-bromoheptan-2-yl)phenyl)-3-oxo-4-phenylcyclopenta-1,4-dien-1-yl)phenyl)-2-(4-(7-bromoheptan-2-yl)phenyl)-4,5-diphenylcyclopenta-2,4-dien-1-one (M13) reacts with p-bis(ethynyl)benzene to yield carbon monoxide and a polymer with a quaterphenylene backbone with a mix of meta and para configurations imparted by the selectivity of a Diels-Alder polymerization, six phenyl side rings and exactly three 7-bromoheptan-2-yl side groups per repeat unit.

In the embodiment, the quaterphenylene polymer derivative 6P4BQP is synthesized by Diets Alder polymerization reaction where 4,4′-(1,4-phenylene)bis(3,5-bis(4-(7-bromoheptan-2-yl)phenyl)-2-phenylcyclopenta-2,4-dien-1-one) (M24) reacts with p-bis(ethynyl)benzene to yield carbon monoxide and a polymer with a quaterphenylene backbone with a mix of meta and para configurations imparted by the selectivity of a Diels-Alder polymerization, six phenyl side rings and exactly four 7-bromoheptan-2-yl side groups per repeat unit.

In the embodiment, following the formation of 6P2BQP the bromine functional groups are substituted with a nitrogen-containing base. FIG. 3 shows a quaterphenylene polymer derivative including exactly two functional groups that is an ammonium group per repeat unit identified as 6P2NQP.

In the embodiment, following the formation of 6P3BQP the bromine functional groups are substituted with a nitrogen-containing base. FIG. 3 shows a quaterphenylene polymer derivative including exactly three functional groups that is an ammonium group per repeat unit identified as 6P3NQP.

In the embodiment, following the formation of 6P4BQP the bromine functional groups are substituted with a nitrogen-containing base. FIG. 3 shows a quaterphenylene polymer derivative including exactly four functional groups that is an ammonium group per repeat unit identified as 6P4NQP.

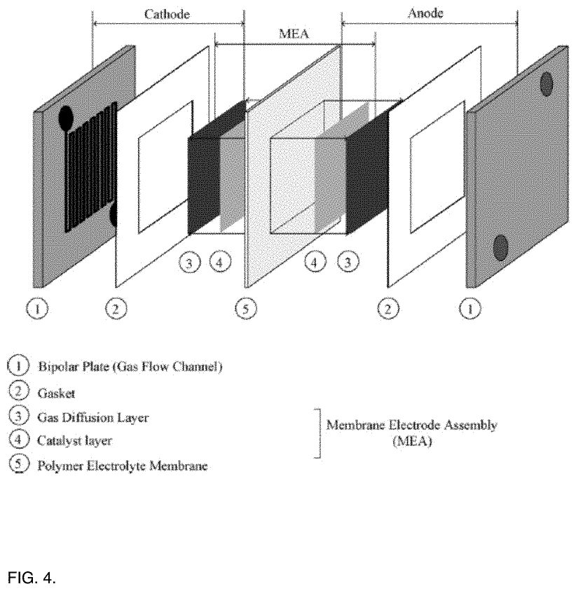

FIG. 4 shows a schematic of a fuel cell membrane electrode assembly (MEA) according to the invention. As can be seen in FIG. 4, the MEA includes a membrane sandwich or disposed between electrodes. The membrane is formed of the disclosed quaterphenylene polymeric derivatives functionalized with the QA groups discussed above. In this embodiment, the membrane has a thickness of 20-50 μm.

The electrodes include a cathode and an anode. The electrodes include the ionomer binder, a support and a catalyst. The ionomer binder can be the quaterphenylene polymeric derivatives functionalized with the QA groups of this disclosure.

The support is carbon particles. In another embodiment, the support may be a porous carbon network or a metallic nanopowder.

The catalyst is a high-surface area metal that can reduce oxygen and oxidize the fuel of interest. In the cathode, the catalyst may be a (metallic nanopowder or finely dispersed metal on a carbon support). In another embodiment, the catalyst may be (Pt, Pd, Ru, Ni, Cu, Fe, Sn, Ag, or some combination of those dispersed on a carbon support). In the anode, the catalyst may be a (metallic nanopowder or finely dispersed metal on a carbon support). In another embodiment, in the anode, the catalyst may be (Pt, Pd, Ru, Ni, Cu, Fe, Sn, Ag, or some combination of those dispersed on a carbon Support). In this exemplary embodiment, the electrodes have a cross sectional thickness of between 1 micron and 10 microns. In another embodiment, the electrodes may have a cross-sectional thickness of between (0.5 and 50 microns).

The ionomer binder serves as both an adhesive to hold the electrodes to the membrane and as a carrier of ions between the membrane and the catalyst. The electrodes must also be designed so that fuel and water can move easily through them to facilitate the electrochemical reactions.

In this exemplary embodiment, the polymer that comprises the membrane and the ionomer binder are the same. In another embodiment, the membrane and/or the ionomer binder may be the same. Thus, the quaterphenylene polymeric derivatives functionalized with the QA groups of this disclosure may be used for either or both the membrane and the ionomer binder.

While the invention has been described with reference to preferred illustrated embodiments, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention is not limited to the particular embodiment disclosed as the best mode contemplated for carrying out this invention, but that the invention will include all embodiments falling within the scope of the appended claims.

What is claimed is:

1. A composition comprising substituted 3,3′-(1,4-phenylene)bis(cyclopenta-2,4-dien-1-one) derivatives as shown in Formula I. 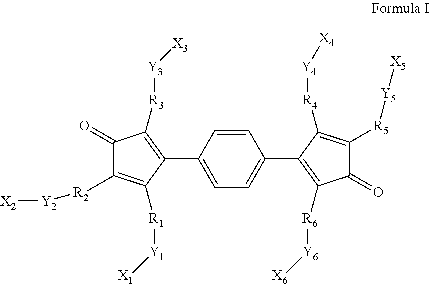
wherein each of R₁, R₂, R₃, R₄, R₅ and R₆ may be the same or different and is H or an unsubstituted or inertly-substituted aromatic moiety;
wherein at least one of Y₁, Y₂, Y₃, Y₄, Y₅ and Y₆ is a monovalent hydrocarbon group including four to 21 carbon atoms that may be the same or different. The monovalent hydrocarbon groups may have a straight chain or a branched chain structure. A straight or branched chain of a monovalent hydrocarbon group may also be interrupted by O, N, or S atoms. wherein each of X₁, X₂, X₃, X₄, X₅ and X₆ may be the same or different and is H or chlorine, bromine and iodine atoms and are present when Y₁, Y₂, Y₃, Y₄, Y₅ and Y₆ is a monovalent hydrocarbon group including four to 21 carbon atoms that may be the same or different. The monovalent hydrocarbon groups may have a straight chain or a branched chain structure. A straight or branched chain of a monovalent hydrocarbon group may also be interrupted by O, N, or S atoms.

2. An oligomer or polymer compound formed by the Diels Alder polymerization reaction between the composition of claim 1 and the p-bis(ethynyl)benzene.

3. The oligomer or polymer compound of claim 2 comprising the same R₁, R₂, R₃, R₄, R₅, R₆, Y₁, Y₂, Y₃, Y₄, Y₅, Y₆, X₁, X₂, X₃, X₄, X₅ and X₆ substituents as the composition of claim 1.

4. The oligomer or polymer compound of claim 3, wherein one or more of X₁, X₂, X₃, X₄, X₅ and X₆ when present comprises a nitrogen containing base.

5. The oligomer or polymer compound of claim 3, wherein one or more of X₁, X₂, X₃, X₄, X₅ and X₆ when present comprises a nitrogen containing resonant stabilized cation group.

6. A membrane comprising the oligomer or polymer compound of claim 5.

7. An electrode comprising the oligomer or polymer compound of claim 5.

8. A method for forming an anion exchange polymer, comprising
A. introducing functional groups to one or more monomer precursors, wherein each of those functional groups comprise one or more monovalent hydrocarbon groups terminated by any of chlorine, bromine, and iodine;
B. polymerizing the functional groups; and
C. substituting a nitrogen-containing base for the functional groups to form a quaterphenylene polymer derivative.

9. The method of claim 8, comprising casting the quaterphenylene polymer derivative in solvent, converting it to hydroxide form, and converting that hydroxide form to an ion pair form with any of phosphoric or phosphinic acids.

10. The method of claim 8, wherein step (B) includes polymerizing the functional groups via a Diels Alder polymerization reaction.
